(19) United States
(12) Patent Application Publication
(10) Pub. No.: US 2021/0196243 A1
(43) Pub. Date: Jul. 1, 2021

(54) MEDICAL IMAGE DIAGNOSTICS SYSTEM AND ULTRASONIC PROBE

(71) Applicant: CANON MEDICAL SYSTEMS CORPORATION, Otawara-shi (JP)

(72) Inventors: Ryota OSUMI, Nasushiobara (JP); Takatoshi OKUMURA, Yaita (JP); Takeshi SATO, Nasushiobara (JP)

(73) Assignee: CANON MEDICAL SYSTEMS CORPORATION, Otawara-shi (JP)

(21) Appl. No.: 17/132,233

(22) Filed: Dec. 23, 2020

(30) Foreign Application Priority Data
Dec. 27, 2019 (JP) 2019-238694
Dec. 23, 2020 (JP) 2020-213218

(51) Int. Cl. A61B 8/00; A61B 8/14

(52) U.S. Cl. CPC A61B 8/565; A61B 8/54; A61B 8/14; A61B 8/4477; A61B 8/4472

(57) ABSTRACT

A medical image diagnostic system according to an embodiment includes a first device, a second device, a communication unit, a first detection circuit, a second detection circuit, and a control circuit. The first device acquires a first signal to be used for diagnosing a subject, acquires a second signal from the first signal, and outputs the first signal or the second signal. The second device includes a generation unit that generates image data by using the first signal or the second signal. The communication unit establishes communication between the second device and the first device and transfers the first signal or the second signal to the second device. The first detection circuit detects a diagnostic status from the first device. The second detection circuit detects a communication status from the communication unit. The control circuit controls at least one of the output from the first device and a communication speed of the communication unit on the basis of the diagnostic status and the communication status.

### CROSS-REFERENCE TO RELATED APPLICATIONS

This application is based upon and claims the benefit of priority from Japanese Patent Application No. 2019-238694, filed on Dec. 27, 2019, and 2020-213218, filed on Dec. 23, 2020; the entire contents of all of which are incorporated herein by reference.

### FIELD

Embodiments described herein relate generally to a medical image diagnostic system and an ultrasonic probe.

### BACKGROUND

In recent years, there has been proposed a medical image diagnostic system that connects a scanner and a computer that performs medical diagnostic data processing, by a network and transfers medical diagnostic data therebetween in a wired or wireless manner.

For example, there has been known an ultrasonic diagnostic system that connects an ultrasonic probe or a portable ultrasonic diagnostic device and an ultrasonic diagnostic image server that performs ultrasonic signal processing, by a network and transfers ultrasonic signals from the ultrasonic probe to the ultrasonic diagnostic image server. In this ultrasonic diagnostic system, when ultrasonic reception signals are transferred from the ultrasonic probe to the ultrasonic diagnostic image server by using wireless communication, the ultrasonic reception signals are sequentially transferred by a batch data transmission scheme after scanning, not in real time, in consideration of communication speed restrictions.

In general, when an imaging mode, an application to be used, and an ultrasonic probe are changed, a data rate required for signal processing, image processing, and transfer is also changed. For example, in the case of the ultrasonic diagnostic system, the imaging mode may be changed from a B mode to a color Doppler mode, an elastography mode, and a contrast enhanced ultrasonography (CEUS) mode within the same examination, and the application may be changed according to the change in the imaging mode. Furthermore, depending on the ultrasonic probe to be used, the specifications of the ultrasonic probe, for example, a center frequency and a frequency band are also changed. Even when the imaging mode, the application to be used, and the ultrasonic probe are changed and the required data rate is changed in this way, it is required that a diagnostic function can be stably continued and maintained as much as possible.

### BRIEF DESCRIPTION OF THE DRAWINGS

FIG. 1 is a diagram illustrating an example of a configuration of a group of ultrasonic diagnostic systems SG according to a first embodiment;

FIG. 2 is a block diagram illustrating an example of configurations of an imaging system S1 and an ultrasonic image server 2 included in the ultrasonic diagnostic system SG;

FIG. 3 is a diagram for explaining an example of a configuration of a reception processing circuit 33;

FIG. 4 is a diagram for explaining an example of the relation between a transfer rate required when a diagnostic status has been changed and a communication status;

FIG. 5 is a diagram illustrating an example of the relation between an upper limit value of a transfer rate changed when a communication status has been changed, and a signal before beamforming R_{Ax} and a signal after beamforming R_{Ay} in a diagnostic status A;

FIG. 6 is a flowchart illustrating an example of the flow of controlling signal output and a communication speed according to a diagnostic status and a communication status;

FIG. 7 is a block diagram illustrating an example of a configuration of the reception processing circuit 33 included in an ultrasonic probe 3 according to a first modification;

FIG. 8 is a diagram for explaining an example of a configuration of an ultrasonic diagnostic system SG according to a third embodiment; and

FIG. 9 is an example of a block diagram illustrating configurations of an ultrasonic probe 3, an ultrasonic diagnostic device 4, and an ultrasonic image server 2 included in the ultrasonic diagnostic system SG according to the third embodiment.

### DETAILED DESCRIPTION

A medical image diagnostic system according to an embodiment includes a first device, a second device, a communication circuit, at least one first detection circuit, at least one second detection circuit, and a control circuit. The first device acquires a first signal to be used for diagnosing a subject, acquires a second signal from the first signal, and outputs the first signal or the second signal. The second device includes a generation unit that generates image data by using the first signal or the second signal. The communication circuit establishes communication between the second device and at least the first device, and transfers the first signal or the second signal that is output from the first device, to the second device. The at least one first detection circuit detects a communication status related to the communication from the communication unit. The at least one second detection circuit detects a diagnostic status related to the subject from the first device. On the basis of the diagnostic status and the communication status, the control circuit controls at least one of the output from the first device and a communication speed of the communication circuit.

Hereinafter, with reference to the drawings, embodiments of a medical image diagnostic system and an ultrasonic probe according to an embodiment will be described in detail. Note that, in the following each embodiment, in order to make the description concrete, a case where the medical image diagnostic system is an ultrasonic diagnostic system will be described as an example. However, the medical image diagnostic system according to the embodiments may be systems other than the ultrasonic diagnostic system.

### First Embodiment

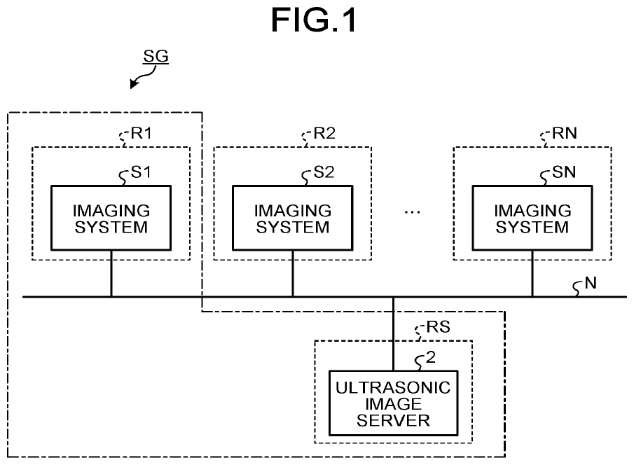

FIG. 1 is a diagram illustrating a configuration of a group of ultrasonic diagnostic systems SG including an ultrasonic diagnostic system SG according to a first embodiment. As illustrated in FIG. 1, the group of ultrasonic diagnostic systems SG is configured by imaging systems S1 to SN provided in a plurality of examination rooms (examination rooms R1 to RN in the example of FIG. 1), respectively, and an ultrasonic image server 2 provided in a server room RS. The imaging systems S1 to SN and the server room RS can communicate with each other via a network N. Note that the server room RS does not necessarily have to be installed in a hospital and may be installed in anywhere as long as it can communicate with the imaging systems S1 to SN via the wired or wireless network N.

Each of the imaging systems S1 to SN includes an ultrasonic probe as a scanner (imaging device), and transfers ultrasonic data acquired using the scanner to the ultrasonic image server 2 via the network N. Furthermore, each of the imaging systems S1 to SN receives ultrasonic image data transferred from the ultrasonic image server 2 and displays an ultrasonic image on a monitor.

Note that, in the present embodiment, the “ultrasonic data” means data based on a reception signal acquired using the ultrasonic probe, and may be either a reception signal before beamforming processing or a reception signal after beamforming processing. Furthermore, the “ultrasonic image data” means image data generated by signal processing using the ultrasonic data.

The ultrasonic image server 2 receives the ultrasonic data that is transferred from each of the imaging systems S1 to SN, via the network N, and generates the ultrasonic image data for each of the imaging systems S1 to SN by using the received ultrasonic data. The ultrasonic image server 2 transfers the generated ultrasonic image data for each of the imaging systems S1 to SN to each of the imaging systems S1 to SN via the network N.

For example, when the ultrasonic data is acquired using the imaging system S1 in the examination room RI, the acquired ultrasonic data is automatically transferred to the ultrasonic image server 2 in the server room RS via the network N. The ultrasonic image server 2 automatically generates the ultrasonic image data by using the acquired ultrasonic data, and transfers the ultrasonic image data to the imaging system S1 of the examination room R1 via the network N. The imaging system S1 of the examination room R1 receives the transferred ultrasonic image data and displays a corresponding ultrasonic image on the monitor.

When a user has performed imaging using the imaging system S1 in the examination room R1, the user can observe the ultrasonic image data which has been generated in the ultrasonic image server 2 in the server room RS, in real time in the examination room R1. Accordingly, a combination of the imaging system S1 and the ultrasonic image server 2 communicably connected via the network N can be referred to as one ultrasonic diagnostic system. Similarly, a combination of each of the other imaging systems S2 to SN and the ultrasonic image server 2 can also be referred to as one ultrasonic diagnostic system. Note that, in the present embodiment, the combination of the imaging system S1 and the ultrasonic image server 2 communicably connected via the network N is referred to as the “ultrasonic diagnostic system SG”.

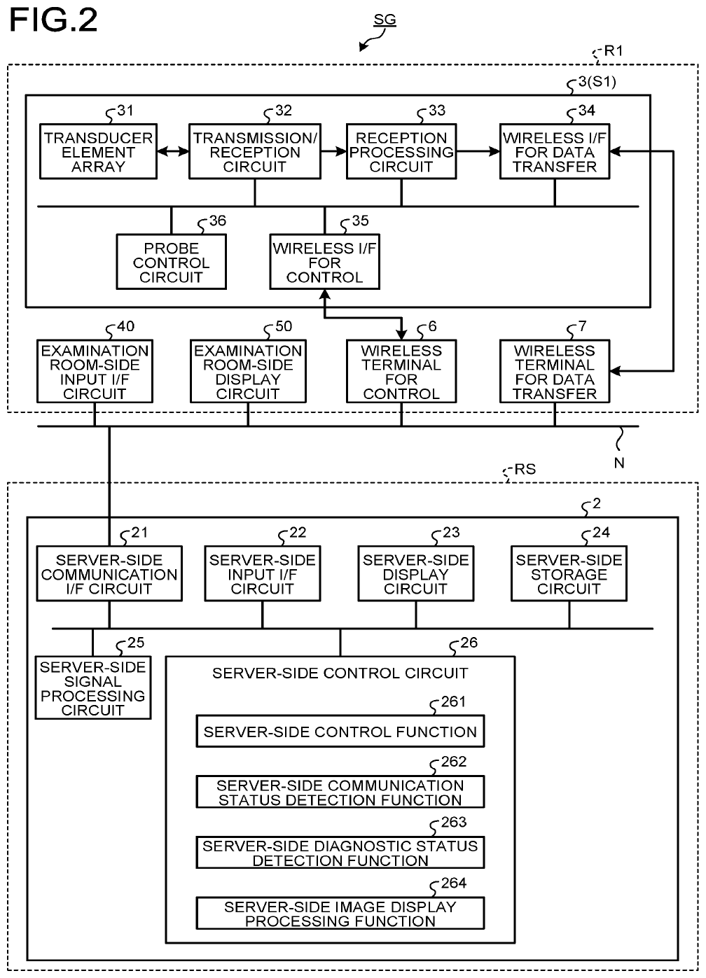

FIG. 2 is a block diagram illustrating the configurations of the imaging system S1 and the ultrasonic image server 2 included in the ultrasonic diagnostic system SG. Hereinafter, the configurations of the imaging system S1 and the ultrasonic image server 2 will be described with reference to FIG. 2. Note that FIG. 2 illustrates the configuration of only the imaging system S1, but since the configurations of the other imaging systems S2 to SN are the same as that of the imaging system S1, a description thereof will be omitted.

First, the imaging system S1 will be described. As illustrated in FIG. 2, the imaging system S1 includes an ultrasonic probe 3, an examination room-side input interface (I/F) circuit 40, an examination room-side display circuit 50, a wireless terminal for control 6, and a wireless terminal for data transfer 7. For example, the ultrasonic probe 3, the examination room-side input I/F circuit 40, and the examination room-side display circuit 50 are disposed around the bed of the examination room R1, and the wireless terminal for control 6 and the wireless terminal for data transfer 7 are installed on the wall or ceiling of the examination room R1.

The ultrasonic probe 3 is a scanner that transmits ultrasonic waves to a subject, receives reflected waves reflected in the subject, and generates ultrasonic data. More specifically, the ultrasonic probe 3 includes a transducer element array 31, a transmission/reception circuit 32, a reception processing circuit 33, a wireless I/F for data transfer 34, a wireless I/F for control 35, and a probe control circuit 36.

The transducer element array 31 has a function of converting a transmission signal applied as an electrical signal via the transmission/reception circuit 32 into ultrasonic waves and transmitting the ultrasonic waves to the subject, and a function of receiving reflected waves generated in the subject by the transmission of the ultrasonic waves, converting the reflected waves into a reception signal being an electrical signal, and outputting the reception signal to each reception channel. The transducer element array 31 has various characteristics and element arrangement modes depending on a diagnosis target, and specifications that affect the data rate of an ultrasonic signal, such as the central frequency and frequency band of the ultrasonic probe 3, are defined by various characteristics and element arrangement modes.

The transmission/reception circuit 32 includes a transmission circuit, a transmission/reception separation circuit, a high-voltage switch, an amplifier, an A/D converter, and a reception buffer memory. The transmission circuit generates a transmission signal for each transmission channel and outputs each transmission signal with a delay time for forming an ultrasonic transmission beam. The transmission signal for each transmission channel output from the transmission circuit is applied to each element of the transducer element array 31 via the transmission/reception separation circuit and the high-voltage switch, so that the ultrasonic transmission beam having directivity is transmitted from the transducer element array 31.

Furthermore, the amplifier of the transmission/reception circuit 32 amplifies the reception signal acquired for each reception channel and outputs the amplified reception signal to the A/D converter. The A/D converter A/D-converts the reception signal for each reception channel that is output from the amplifier as an analog signal, into a digital reception signal. The reception signals as a plurality of radio frequency (RF) waves after the A/D conversion, corresponding to the transducer element array 31, are stored in the reception buffer memory.

The reception processing circuit 33 performs reception processing, such as data compression and beamforming, on the reception signals received from the transmission/reception circuit 32. Furthermore, the reception processing circuit 33 selectively outputs ultrasonic data before the beamforming processing and ultrasonic data after the beamforming processing.

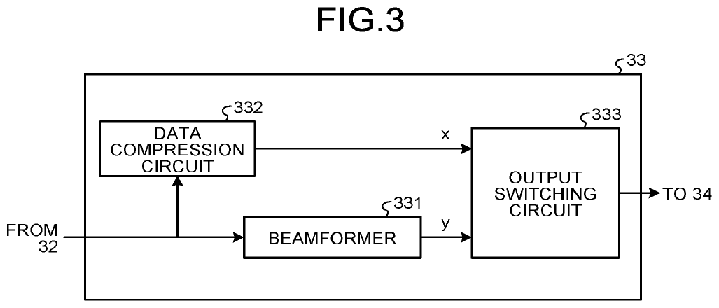

FIG. 3 is a diagram for explaining the configuration of the reception processing circuit 33. As illustrated in FIG. 3, the reception processing circuit 33 includes a beamformer 331, a data compression circuit 332, and an output switching circuit 333.

The beamformer 331 performs the beamforming processing on the reception signals stored in the reception buffer memory of the transmission/reception circuit 32.

Examples of the beamforming processing include delay-and-sum beamforming and adaptive beamforming. The delay-and-sum beamforming is a process of giving a reception delay time for each reception channel to each reception signal for addition. Furthermore, the adaptive beamforming is a method of performing delay time correction in consideration of a sound velocity distribution inside a subject. In general, according to the adaptive beamforming, high image quality can be expected for various subjects. On the other hand, the amount of data processed is very large and considerable power is required for real-time processing. Accordingly, when Performing the adaptive beamforming with an ultrasonic probe that requires miniaturization, release of generated thermal energy becomes problematic.

In the present embodiment, it is assumed that the beamformer 331 performs the delay-and-sum beamforming, in order to make the description concrete. However, beamforming performed by the beamformer 331 is not limited to the delay-and-sum beamforming, and the adaptive beamforming can also be adopted as needed.

The data compression circuit 332 performs data compression processing on the reception signals stored in the reception buffer memory of the transmission/reception circuit 32. The data compression uses the fact that reception signals are similar between adjacent reception channels. In the present embodiment, lossless compression with a compression rate of about ⅓ is assumed, and the compression rate is, for example, 8.3 Gbps depending on the conditions of the number of reception channels, a bit depth, and a reception signal frequency.

Note that when the beamformer 331 performs no beamforming, if it is assumed that the number of reception channels is 64, the bit depth of the reception signal is 10 bits, and the frequency of the reception signal is 40 MHz, a data rate of 25 Gbps 64 channels×10 bits×40 MHz) is required in order to perform real-time transfer. On the other hand, for example, in IEEE802.11ay for Wi-Fi (registered trademark) communication among wireless communication standards, a data rate of a maximum of 100 Gbps is assumed, but in the present embodiment, in consideration of various communication statuses, a data compression unit 302 compresses a signal that is not subjected to beamforming.

On the other hand, according to the communication standard used in the ultrasonic diagnostic system SG, when there is enough room to transfer a signal that is not subjected to beamforming, without compression, an uncompressed reception signal before the beamforming processing may be output from the data compression circuit 332 to the output switching circuit 333. Moreover, the compression processing in the data compression circuit 332 may also be ON/OFF-controlled depending on the situation.

The output switching circuit 333 outputs any one of the reception signal after the beamforming processing received from the beamformer 331 and the reception signal after the data compression processing received from the data compression circuit 332, as ultrasonic data in response to a control signal from the ultrasonic image server 2. By controlling the switching of the output from the output switching circuit 333, it is possible to select the transfer rate related to the data transfer from the imaging system S1 to the ultrasonic image server 2.

Returning back to FIG. 2, the wireless I/F for data transfer 34 converts the ultrasonic data acquired from the reception processing circuit 33 into a wireless signal conforming to the wireless communication standard and sends the wireless signal to the wireless terminal for data transfer 7. Note that, in order to simplify the description, the present embodiment adopts, as an example, a case where the communication standard of the wireless terminal for data transfer 7 and the wireless I/F for data transfer 34 is single.

The wireless I/F for control 35 converts a control signal to be transferred to the ultrasonic image server 2 among probe control signals generated by the probe control circuit 36 into a wireless signal conforming to the wireless communication standard, and sends the wireless signal to the wireless terminal for control 6.

Note that, in the present embodiment, it is assumed that wireless communication interfaces of the ultrasonic reception signal and the probe control signal are made independent and the signals are transferred using separate communication standards such that the signals do not affect each other as much as possible. For example, the ultrasonic data is transferred by Wi-Fi (registered trademark) and the probe control signal is transferred by Bluetooth (registered trademark). Other communication standards, for example, ultra-wide band (UWB) or the like may also be used.

The probe control circuit 36 is a processor that controls the transmission/reception circuit 32, the reception processing circuit 33, the wireless I/F for data transfer 34, and the wireless I/F for control 35.

The examination room-side input I/F circuit 40 accepts various input operations from a user, converts the accepted input operations into electrical signals, and outputs the electrical signals to a server-side communication I/F circuit 21. For example, the examination room-side input I/F circuit 40 accepts ultrasonic data collection conditions, image processing conditions related to ultrasonic images, image display conditions, and the like from the user. Specifically, the examination room-side input I/F circuit 40 is implemented by a track ball, a switch button, a mouse, a keyboard, a touch pad for performing an input operation by touching an operation screen thereof, a touch pad for performing an input operation by touching an operation screen thereof, a touchscreen in which a display screen and the touch pad are integrally formed, a non-contact input I/F circuit using an optical sensor, a touch panel display in which a voice input I/F circuit, the display screen, and the touch pad are integrally formed, or the like.

Note that the examination room-side input I/F circuit 40 is not limited only to those provided with physical operating parts such as the mouse and the keyboard. For example, an electrical signal processing circuit that receives an electrical signal corresponding to an input operation from an external input device provided separately from the imaging device and outputs the electrical signal to the control circuit, is also included in a server-side input I/F circuit 22.

The examination room-side display circuit 50 is a liquid crystal display or a cathode ray tube (CRT) display. The examination room-side display circuit 50 is a monitor referred by the user and displays various information. For example, the examination room-side display circuit 50 outputs an ultrasonic image based on the ultrasonic image data received from the ultrasonic image server 2, a graphical user interface (GUI) for accepting various operations from the user, and the like. Note that the ultrasonic image displayed on the examination room-side display circuit 50 is sent from the ultrasonic image server 2 via the network N. Therefore, the examination room-side display circuit 50 includes a wired communication interface.

The wireless terminal for control 6 establishes communication between the ultrasonic image server 2 and the ultrasonic probe 3, and transmits/receives the control signal and the like. The wireless terminal for control 6 is connected to the wireless I/F for control 35, and transmits/receives the probe control signal converted into the wireless signal conforming to the wireless communication standard. For example, the wireless terminal for control 6 transmits the probe control signal received from the wireless I/F for control 35 to the ultrasonic image server 2 via the network N. Furthermore, the wireless terminal for control 6 transmits the probe control signal received from the ultrasonic image server 2 via the network N to the wireless I/F for control 35.

The wireless terminal for data transfer 7 establishes communication between the ultrasonic image server 2 and the ultrasonic probe 3, and transfers the ultrasonic data output from the ultrasonic probe 3 to the ultrasonic image server 2. The wireless terminal for data transfer 7 is connected to the wireless I/F for data transfer 34, and transmits/receives the ultrasonic data converted into the wireless signal conforming to the wireless communication standard. For example, the wireless terminal for data transfer 7 transmits the ultrasonic data received from the wireless I/F for data transfer 34 to the ultrasonic image server 2 via the network N. Furthermore, the wireless terminal for data transfer 7 transmits the ultrasonic data and the ultrasonic image data received from the ultrasonic image server 2 via the network N to the wireless I/F for data transfer 34.

Note that, in the present embodiment, in order to make the description concrete, it is assumed that the wireless communication standard between the wireless terminal for control 6 and the wireless I/F for control 35 is different from that between the wireless terminal for data transfer 7 and the wireless I/F for data transfer 34.

Next, the configuration of the ultrasonic image server 2 will be described. The ultrasonic image server 2 generates the ultrasonic image by using the ultrasonic data acquired by the ultrasonic probe 3. As illustrated in FIG. 2, the ultrasonic image server 2 includes the server-side communication I/F circuit 21, the server-side input I/F circuit 22, a server-side display circuit 23, a server-side storage circuit 24, a server-side signal processing circuit 25, and a server-side control circuit 26.

The server-side communication I/F circuit 21 performs a communication operation with an external device via the network according to a predetermined communication standard.

Since the server-side input I/F circuit 22 has the same configuration as that of the examination room-side input I/F circuit 40, a description thereof will be omitted.

Since the server-side display circuit 23 has the same configuration as that of the examination room-side display circuit 50, a description thereof will be omitted.

The server-side storage circuit 24 is configured by a semiconductor memory device such as a random-access memory (RAM) and a flash memory, a hard disk, an optical disc, and the like. The server-side storage circuit 24 may be configured by a portable medium such as a universal serial bus (USB) memory and a digital video disc (DVD). The server-side storage circuit 24 can store therein various images or information, the ultrasonic data transferred from the ultrasonic probe 3, the output of the server-side signal processing circuit 25, and the like. The form of the storage includes a case where live information is temporarily stored and a case of storage for long-term recording for evidence of acquired patient information. Furthermore, the server-side storage circuit 24 stores therein diagnostic information (for example, patient IDs, doctor's opinions, and the like), and various data such as diagnostic protocols and various body marks.

Furthermore, the storage circuit 20 stores therein various processing programs (an operating system (OS), and the like in addition to application programs) used by the server-side control circuit 26, data required for executing computer programs, volume data, and medical images. Furthermore, the OS may also include a GUI that makes frequent use of graphics for displaying information on the server-side display circuit 23 for an operator and allows the server-side input I/F circuit 22 to perform basic operations.

The server-side signal processing circuit 25 generates the ultrasonic image by using the ultrasonic data acquired from the ultrasonic probe 3 via the wireless I/F for data transfer 34, the wireless terminal for data transfer 7, and the server-side communication I/F circuit 21. Specifically, the server-side signal processing circuit 25 performs the adaptive beamforming or the delay-and-sum beamforming on ultrasonic data before beamforming processing, thereby generating ultrasonic data after the beamforming processing. Furthermore, the server-side signal processing circuit 25 performs each of phase detection, envelope detection, and logarithmic compression processing on the ultrasonic data after the beamforming processing, thereby generating the ultrasonic image corresponding to a B mode. Moreover, the server-side signal processing circuit 25 generates ultrasonic images corresponding to various imaging modes such as color Doppler, contrast, shear wave elastography, and attenuation.

Note that various processes performed by the server-side signal processing circuit 25 can be processed by software as an ultrasonic image processing program using a high-speed processor, and particularly, for beamforming, the aforementioned adaptive beamforming can be applied.

The server-side control circuit 26 is a processor that reads and executes the computer programs from the server-side storage circuit 24, thereby performing functions corresponding to the respective computer programs. The server-side control circuit 26 reads various control programs stored in the server-side storage circuit 24, thereby implementing a server-side control function 261, a server-side communication status detection function 262, a server-side diagnostic status detection function 263, and a server-side image display processing function 264, and comprehensively controlling processing operations in the server-side communication I/F circuit 21, the server-side input I/F circuit 22, the server-side display circuit 23, and the server-side storage circuit 24. In other words, the server-side control circuit 26 in the state of having read the respective computer programs has the respective functions illustrated in the server-side control circuit 26 of FIG. 2.

Furthermore, the server-side control function 261 controls the entire processing of the ultrasonic image server 2. Specifically, on the basis of various setting requests input from an operator via the server-side input I/F circuit 22, various control programs, and various data, the server-side control function 261 controls the server-side communication I/F circuit 21, the server-side display circuit 23, the server-side storage circuit 24, and the server-side signal processing circuit 25.

Furthermore, on the basis of a communication status and a diagnostic status (examination status), the server-side control function 261 controls at least one of the output switching circuit 333 of the reception processing circuit 33 and a communication speed between the wireless I/F for data transfer 34 and the wireless terminal for data transfer 7.

The “communication status” means a maximum communication speed which can be currently achieved in communication between the ultrasonic probe 3 and the ultrasonic image server 2. The maximum communication speed in the communication between the ultrasonic probe 3 and the ultrasonic image server 2 may be changed according to the number of other ultrasonic probes to be Wi-Fi connected to the wireless terminal for data transfer 7 when the wireless I/F for data transfer 34 and the wireless terminal for data transfer 7 are connected to each other by Wi-Fi, and a primary malfunction of the wireless terminal for data transfer 7, for example. Furthermore, in the ultrasonic probe 3 using a battery as a power supply means, the maximum communication speed in the communication between the ultrasonic probe 3 and the ultrasonic image server 2 may be controlled according to the remaining level of the battery, such that a communication speed slows down when the remaining level of the battery falls below a certain level, for example. The communication status means the maximum communication speed that can be achieved in such an environment where the communication speed may be changed.

Furthermore, the “diagnostic status” is information indicating that what kind of diagnosis is currently being performed using the ultrasonic probe 3. Specifically, the diagnostic status is information including the specifications (a center frequency, a frequency band, and the like) of the ultrasonic probe 3, an imaging mode currently set in the ultrasonic image server 2, imaging conditions (a frame rate in ultrasonic transmission/reception, the number of beams per frame, and the like), and the type of an application. By grasping the diagnostic status, it is possible to calculate a data rate required for data transfer from the ultrasonic probe 3 to the ultrasonic image server 2 in the current situation.

Furthermore, on the basis of the communication status and the diagnostic status, the server-side control function 261 switches the output switching circuit 333 between an x side and a y side. Note that the server-side control function 261 is an example of a control circuit.

The server-side communication status detection function 262 monitors a communication status between the wireless I/F for data transfer 34 and the wireless terminal for data transfer 7 and a communication status between the wireless terminal for data transfer 7 and the server-side communication I/F circuit 21 of the ultrasonic image server 2, and measures an upper limit of a data rate that can be achieved by transfer between the ultrasonic probe 3 and the ultrasonic image server 2. A specific example of the communication status monitoring performed by the server-side communication status detection function 262 includes threshold setting of detecting the average reception intensity of radio waves (Enrtgy Detect) among clear channel assessment (CCA) functions in the Wi-Fi wireless communication standard IEEE802.11. Note that the server-side communication status detection function 262 is an example of a first detection circuit.

The server-side diagnostic status detection function 263 detects the diagnostic status from the specifications (the center frequency, the frequency band, and the like) of the ultrasonic probe 3, the frame rate in the ultrasonic transmission/reception, the number of beams per frame, the imaging mode currently set in the ultrasonic image server 2, the imaging conditions, and the type of the application, and calculates the data rate required for the transfer from the ultrasonic probe 3 to the ultrasonic image server 2. Note that the server-side diagnostic status detection function 263 is an example of a second detection circuit.

The server-side image display processing function 264 converts the B mode and other scanning systems into a scanning system suitable for display (scan conversion), and generates an ultrasonic diagnostic image as a display image. Information indicating the combination, juxtaposition, and display position of image information, various information for assisting the operation of an ultrasonic diagnostic device, and supplementary information required for ultrasonic diagnosis such as patient information are also generated together with the ultrasonic diagnostic image.

Control of signal output according to diagnostic status and communication status

Next, the control of the signal output according to the diagnostic status and the communication status that is performed in the ultrasonic diagnostic system SG according to an embodiment will be described.

In general, the data rate required for the ultrasonic data processing performed in the ultrasonic probe 3 and the ultrasonic image server 2 depends on the diagnostic status such as the specifications of the ultrasonic probe 3 to be used and the imaging mode. On the other hand, by achieving the communication status in real time, it is possible to grasp the maximum communication speed that can be currently achieved in the communication between the ultrasonic probe 3 and the ultrasonic image server 2.

That is, the server-side control function 261 compares the diagnostic status with the communication status, thereby determining whether data transfer from the ultrasonic probe 3 to the ultrasonic image server 2 can be implemented as is or data transfer at a higher rate than the present rate can be implemented. On the basis of the determination result, the server-side control function 261 controls at least one of the output switching circuit 333 of the reception processing circuit 33 and the communication speed between the wireless I/F for data transfer 34 and the wireless terminal for data transfer 7 on the basis of the communication status and the diagnostic status.

First, it is assumed that the communication status is constant and the diagnostic status is changed, that is, a case where the data rate required for transfer from the ultrasonic probe 3 to the ultrasonic image server 2 is changed by a change in the imaging mode. Such a case occurs when the ultrasonic probe 3 is changed to another type of ultrasonic probe and the specifications of the ultrasonic probe, for example, the central frequency and the frequency band are changed.

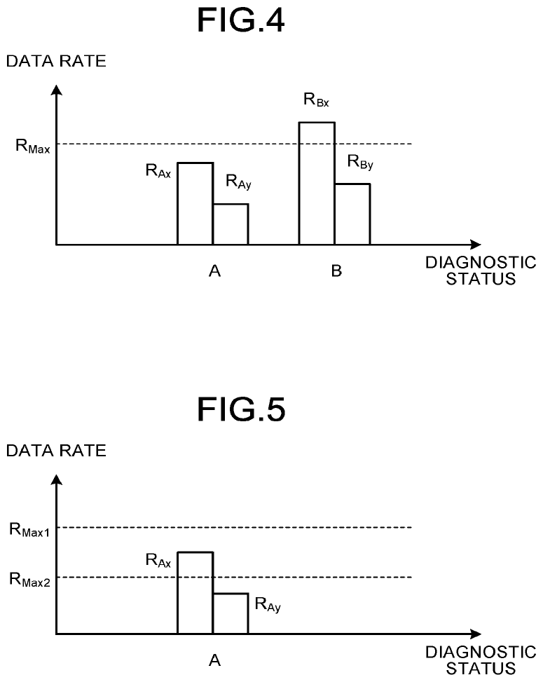

FIG. 4 is a diagram for explaining the relation between a transfer rate required when the diagnostic status has been changed and the communication status. As illustrated in FIG. 4, it is assumed that in a diagnostic status A, the required transfer rate is R_{Ax} for a signal before beamforming (that is, the output switching circuit 333 is switched to x) and is R_{Ay} for a signal after beamforming (that is, the output switching circuit 333 is switched to y). Furthermore, it is assumed that in a diagnostic status B, the required transfer rate is R_{Bx} for a signal before beamforming (that is, the output switching circuit 333 is switched to x) and is R_{By} for a signal after beamforming (that is, the output switching circuit 333 is switched to y).

In the diagnostic status A, the signal before beamforming R_{Ax} and the signal after beamforming R_{Ay} have a value lower than an upper limit R_{Max} due to the communication status. Accordingly, in the diagnostic status A, it is Possible to transfer both the signal before beamforming R_{Ax} and the signal after beamforming R_{Ay}. In such a case, when the ultrasonic image server 2 performs the adaptive beamforming, since high image quality is obtained, the signal before beamforming R_{Ax} is transferred and the switching of the output switching circuit 333 is set to the x side.

On the other hand, in FIG. 4, in the diagnostic status B, the signal before beamforming R_{Bx} has a value higher than the upper limit R_{Max} and the signal after beamforming R_{By} has a value lower than the upper limit R_{MAX} due to the communication status. Accordingly, in the diagnostic status B, it is possible to transfer the signal after beamforming R_{By} at R_{Max} and it is not possible to transfer the signal after beamforming R_{By} at R_{Max}. Accordingly, in the diagnostic status B, the switching of the output switching circuit 333 is set to the y side and the signal after beamforming R_{By} is transferred from the ultrasonic probe 3 to the ultrasonic image server 2. With this, although the image quality is not as high as that of the adaptive beamforming, image quality equivalent to that of a popular ultrasonic diagnostic device can be guaranteed using the beamformer 331 embedded in the ultrasonic probe 3.

Next, it is conceivable that the diagnostic status is fixed and the communication status is changed. That is, when there is no change in the imaging mode or the application and the data rate required for the ultrasonic data processing is constant, the communication status may be changed depending on the environment.

For example, when there is a Wi-Fi terminal in the vicinity of the ultrasonic probe 3 capable of wirelessly transferring an ultrasonic signal and the Wi-Fi terminal can be connected to a plurality of ultrasonic probes, a communication speed from the ultrasonic probe 3 to the ultrasonic image server 2 is changed by the number of connections of the ultrasonic probes to the Wi-Fi terminal. Furthermore, it is also conceivable that communication is not possible between the ultrasonic probe 3 and the ultrasonic image server 2 due to a temporary malfunction of the Wi-Fi terminal. Moreover, for example, when a plurality of streams of wireless communication are secured between the ultrasonic probe 3 and the wireless terminal for data transfer 7 in order to transfer the ultrasonic reception signal, one of the streams may interfere with the signal due to an influence of another communication device and the communication status may be worsened (deteriorates). In these cases, the communication status is also changed according to a change in the communication speed. However, even when the communication status is changed in this way, the ultrasonic diagnostic system SG is required to be continued and maintained as stable as possible.

FIG. 5 is a diagram illustrating the relation between an upper limit value of a transfer rate changed when the communication status has been changed and the signal before beamforming R_{Ax} and the signal after beamforming R_{Ay} in the diagnostic status A. As illustrated in FIG. 5, it is assumed that in the diagnostic status A, the required transfer rate is R_{Ax} for the signal before beamforming (that is, the output switching circuit 333 is switched to x) and is R_{Ay} for the signal after beamforming (that is, the output switching circuit 333 is switched to y).

When the communication status can secure an upper limit R_{Max1} of the transfer rate, it is possible to transfer both the signal before beamforming R_{Ax} and the signal after beamforming R_{Ay} from the ultrasonic probe 3 to the ultrasonic image server 2. In such a communication status, when the ultrasonic image server 2 performs the adaptive beamforming, since high image quality is obtained, the signal before beamforming R_{Ax} is transferred and the switching of the output switching circuit 333 is set to the x side.

On the other hand, it is assumed that the upper limit of the transfer rate falls from R_{Max1} to R_{Max2} and the communication status is worsened. In such a case, since the upper limit R_{Max2} of the transfer rate becomes less than the signal before beamforming R_{Ax}, it is not possible to transfer the signal before beamforming. Therefore, the switching of the output switching circuit 333 is set to the y side and the signal after beamforming R_{By} is transferred from the ultrasonic probe 3 to the ultrasonic image server 2. With this, although the image quality is not as high as that of the adaptive beamforming, image quality equivalent to that of the popular ultrasonic diagnostic device and real-time image generation can be guaranteed using the beamformer 331 embedded in the ultrasonic probe 3. Note that when the upper limit of the transfer rate is returned from R_{Max2} to R_{Max 1} by continuously detecting the communication status, the switching of the output switching circuit 333 is set from the y side to the x side and the signal after beamforming R_{Bx} can also be transferred from the ultrasonic probe 3 to the ultrasonic image server 2.

Control of communication speed according to diagnostic status and communication status

Next, the control of the communication speed according to the diagnostic status and the communication status that is performed in the ultrasonic diagnostic system SG according to the embodiment will be described. Note that the control of the communication speed according to the diagnostic status and the communication status to be described below can also be combined with the control of the signal output according to the diagnostic status and the communication status described above.

In the ultrasonic diagnosis, when the diagnostic process is not successful, a heavy burden may be imposed on a patient and a medical facility. For example, in one-time contrast enhanced ultrasonography diagnosis where contrast media are expensive, the diagnostic cost is relatively high. In such a diagnosis, a high priority needs to be set in the stability of the diagnostic process. Accordingly, it is particularly required to secure various conditions of the diagnostic system including the transfer rate of the ultrasonic reception signal without depending on a change in the communication status.

In this regard, the ultrasonic diagnostic system SG according to the present embodiment sets the priority of diagnosis according to a diagnostic mode and changes the allocation of a communication band used by the wireless terminal for data transfer 7 according to the set priority, thereby controlling the communication speed between the ultrasonic probe 3 and the ultrasonic image server 2.

That is, when the server-side control function 261 of the server-side control circuit 26 performs a high priority diagnosis as a result of detecting the diagnostic status by the server-side diagnostic status detection function 263, the wireless terminal for data transfer 7 installed in the examination room R1 performs exclusive control so as to connect only a specific ultrasonic probe 3. As a consequence, the wireless terminal for data transfer 7 always allocates all communication bands and streams to the ultrasonic probe 3. Furthermore, the connection between the wireless terminal for data transfer 7 and the ultrasonic probe 3 can also be referred to as a point-to-point (P2P) connection.

Note that as for the priority of the diagnosis, instead of setting the presence or absence of the priority for each diagnosis item, when the priority of an application related to the diagnosis item is weighted numerically and a plurality of applications are applied at the same time, communication resources may be preferentially allocated to an application with the highest priority.

Furthermore, in addition to the exclusive control of allocating all bands to an application with a high priority, the ratio of a band to be allocated can also be adjusted according to the priority such as allocating many bands to the application with a high priority.

In this way, the wireless terminal for data transfer 7 is not connected to other ultrasonic probes with a low priority in the examination room R1 or allocates many bands to ultrasonic probes with a high priority. Accordingly, it is possible to allocate communication resources to high-priority diagnosis as much as possible without being affected by surrounding communication terminals as much as possible. As a consequence, when the high-priority diagnosis is performed, it is possible to secure various conditions of the diagnostic system including the transfer rate of the ultrasonic reception signal without depending on a change in the communication status.

Operation

Next, the flow of controlling the signal output and the communication speed according to the diagnostic status and the communication status that is performed in the ultrasonic diagnostic system SG according to an embodiment will be described.

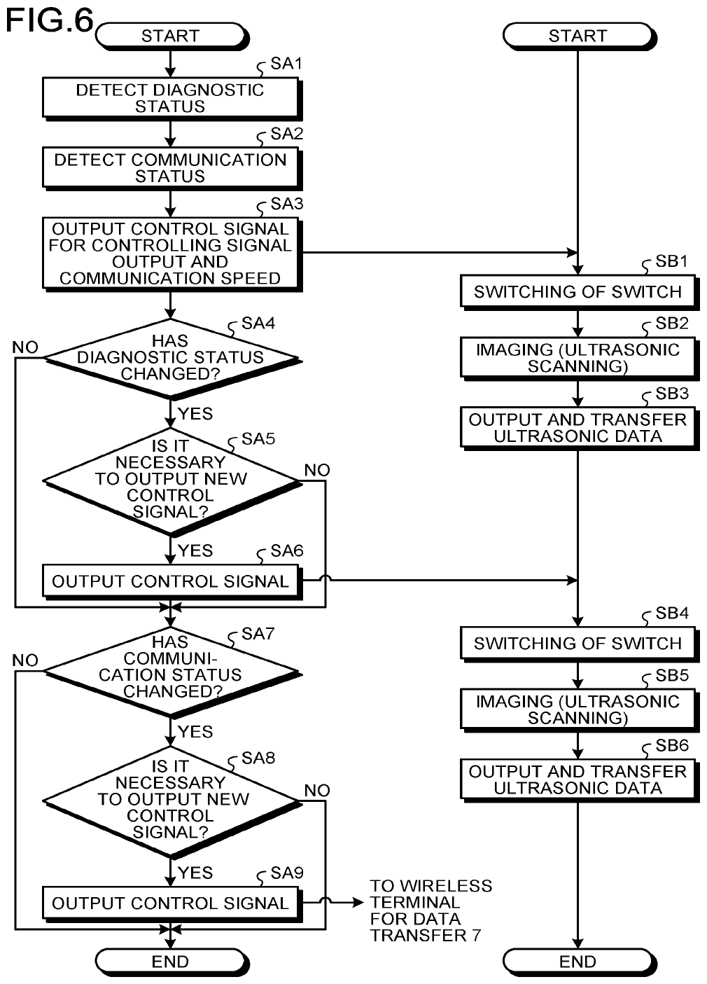

FIG. 6 is a flowchart illustrating the flow of controlling the signal output and the communication speed according to the diagnostic status and the communication status. Note that in FIG. 6, the left column thereof illustrates the processing flow in the ultrasonic image server 2 and the right column thereof illustrates the processing flow in the ultrasonic probe 3.

As illustrated in FIG. 6, the server-side control circuit 26 detects the diagnostic status by the server-side diagnostic status detection function 263 (step SA1). Furthermore, the server-side control circuit 26 detects the communication status by the server-side communication status detection function 262 (step SA2). Note that the order of the processes of steps SA1 and SA2 may be changed.

Next, on the basis of the detected diagnostic status and communication status, the server-side control circuit 26 outputs, with the server-side control function 261, a control signal for switching the output of the output switching circuit 333 of the ultrasonic probe 3 to x or y and a control signal for setting the band allocation of the wireless terminal for data transfer 7 (step SA3). For example, when the imaging mode has been set to the B mode, the server-side control circuit 26 outputs, with the server-side control function 261, a control signal for switching the output of the output switching circuit 333 of the ultrasonic probe 3 to the x side and a control signal for setting the band of the wireless terminal for data transfer 7 to be allocated to a normal band.

The probe control circuit 36 switches the output of the output switching circuit 333 to the x side in response to the control signal from the ultrasonic image server 2 (step SB1). Furthermore, the wireless terminal for data transfer 7 controls the band allocation in response to the control signal from the ultrasonic image server 2.

The probe control circuit 36 performs ultrasonic scanning (step SB2). A reception signal before beamforming processing for each channel obtained by the ultrasonic scanning is output from the reception processing circuit 33 as ultrasonic data and is transferred to the ultrasonic image server 2 via the wireless terminal for data transfer 7 (step SB3).

The server-side control circuit 26 determines with the server-side diagnostic status detection function 263 whether the diagnostic status has changed (step SA4). When the diagnostic status has changed (Yes at step SA4), the server-side control circuit 26 determines whether it is necessary to output, by the server-side control function 261, a new control signal for switching the output of the output switching circuit 333 of the ultrasonic probe 3 to x or y and a new control signal for setting the band allocation of the wireless terminal for data transfer 7 (step SA5).

As a result of the determination, when it is determined that the new control signals need to be generated (Yes at step SA5), the server-side control circuit 26 outputs, with the server-side control function 261, the new control signal for switching the output of the output switching circuit 333 of the ultrasonic probe 3 and the new control signal for changing the band allocation of the wireless terminal for data transfer 7 (step SA6). For example, when the imaging mode is changed from the B mode to the color Doppler mode, the server-side control circuit 26 outputs, with the server-side control function 261, the new control signal for switching the output of the output switching circuit 333 of the ultrasonic probe 3 from the x side to the y side.

The probe control circuit 36 switches the output of the output switching circuit 333 from the x side to the y side in response to the control signals from the ultrasonic image server 2 (step SB4), and performs ultrasonic scanning (step SB5). A reception signal after beamforming processing for each channel obtained by the ultrasonic scanning is output from the reception processing circuit 33 as ultrasonic data and is transferred to the ultrasonic image server 2 via the wireless terminal for data transfer 7 (step SB6).

On the other hand, as a result of the determination in steps SA4 and SA5, when it is determined that the new control signals do not need to be generated (No at steps SA4 and SA5), the server-side control circuit 26 proceeds to step SA7.

The server-side control circuit 26 determines with the server-side communication status detection function 262 whether the communication status has changed (step SA7). When the communication status has changed (Yes at step SA7), the server-side control circuit 26 determines whether it is necessary to output, by the server-side control function 261, a new control signal for switching the output of the output switching circuit 333 of the ultrasonic probe 3 to x or y and a new control signal for setting the band allocation of the wireless terminal for data transfer 7 (step SA8).

As a result of the determination, when it is determined that the new control signals need to be generated (Yes at step SA8), the server-side control circuit 26 outputs, with the server-side control function 261, a control signal for further increasing the ratio of a band to be used for the communication between the ultrasonic probe 3 and the ultrasonic image server 2, for example (step SA9). The wireless terminal for data transfer 7 changes the band allocation in response to the control signal from the ultrasonic image server 2.

Note that the respective processes of steps SA4 to SA9 are repeatedly performed during the ultrasonic scanning.

The ultrasonic diagnostic system according to the present embodiment described above includes the ultrasonic probe 3 as the first device, the ultrasonic image server 2 as the second device, the wireless terminal for data transfer 7 as the communication unit, the server-side communication status detection function 262 as the first detection circuit, the server-side diagnostic status detection function 263 as the second detection circuit, and the server-side control function 261 as the control circuit. The ultrasonic probe 3 acquires a reception signal before beamforming processing as a first signal to be used for diagnosing a subject, acquires a reception signal after beamforming processing as a second signal from the reception signal before beamforming processing, and outputs the reception signal before beamforming processing or the reception signal after beamforming processing. The ultrasonic image server 2 generates image data by using the reception signal before beamforming processing or the reception signal after beamforming processing. The wireless terminal for data transfer 7 establishes communication between the ultrasonic image server 2 and at least the ultrasonic probe 3, and transfers the reception signal before beamforming processing or the reception signal after beamforming processing that is output from the ultrasonic probe 3, to the ultrasonic image server 2. The server-side communication status detection function 262 of the ultrasonic image server 2 detects a communication status related to the communication from the wireless terminal for data transfer 7. The server-side diagnostic status detection function 263 detects a diagnostic status related to the subject from the ultrasonic probe 3. The server-side control function 261 controls at least one of the output from the ultrasonic probe 3 and the communication speed of the wireless terminal for data transfer 7 on the basis of the diagnostic status and the communication status.

Accordingly, by comparing the diagnostic status with the communication status, it is possible to determine whether data transfer from the ultrasonic probe 3 to the ultrasonic image server 2 can be implemented as is or data transfer at a higher rate than the present rate can be implemented. For example, when the current maximum communication speed is high and the reception signal before beamforming processing can be transferred, the server-side control function 261 transfers the reception signal before beamforming processing from the ultrasonic probe 3 to the ultrasonic image server 2. The ultrasonic image server 2 can perform the adaptive beamforming by using the reception signal before beamforming processing, thereby generating an ultrasonic image with high image quality. Furthermore, for example, when the current maximum communication speed is relatively low and it is not possible to transfer the reception signal before beamforming processing, the server-side control function 261 transfers the reception signal after beamforming processing from the ultrasonic probe 3 to the ultrasonic image server 2. The ultrasonic image server 2 can generate an ultrasonic image with image quality equivalent to that of the popular ultrasonic diagnostic device, by using the reception signal after beamforming processing. Furthermore, for example, other ultrasonic probes with a low priority of diagnosis is not connected or it is possible to allocate many bands to ultrasonic probes with a high priority. Accordingly, it is possible to allocate communication resources to high-priority diagnosis as much as possible without being affected by surrounding communication terminals as much as possible.

As a consequence, even when the diagnostic status or the communication status is changed, it is possible to continue and maintain the diagnostic function as much as possible and to implement a stabler operation than in the related art.

First Modification

In the aforementioned embodiment, as a method of lowering the transfer rate of the ultrasonic reception signal depending on the communication status, a method in which data to be transferred from the ultrasonic probe 3 to the ultrasonic image server 2 is switched from pre-beamforming data to post-beamforming data has been exemplified. On the other hand, as another method of lowering the transfer rate of the ultrasonic reception signal depending on the communication status, it is possible to adopt a method in which the ultrasonic probe 3 thins out ultrasonic data and transfers the thinned ultrasonic data.

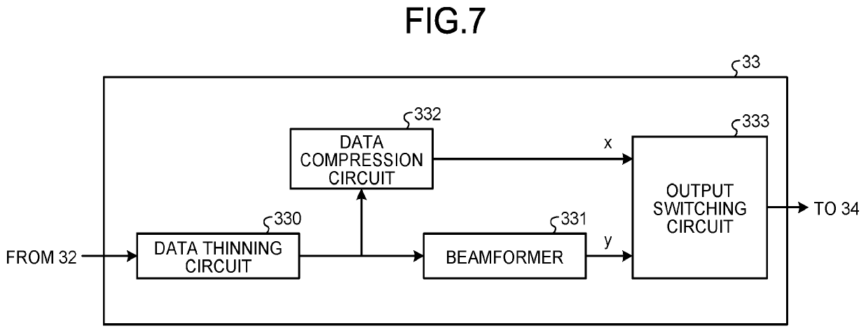

FIG. 7 is a block diagram illustrating the configuration of the reception processing circuit 33 included in the ultrasonic probe 3 according to the first modification. As illustrated in FIG. 7, the reception processing circuit 33 includes a data thinning circuit 330 in addition to the configuration illustrated in FIG. 3.

The data thinning circuit 330 performs data thinning processing on the ultrasonic data from the transmission/reception circuit 32, and outputs the thinned ultrasonic data to the beamformer 331 and the data compression circuit 332. That is, the data thinning circuit 330 performs sub-array processing in which reception signals are phase-corrected and added for each of a plurality of channels, frame rate reduction in which reception signals are added for each of a plurality of time phases, and the like on the ultrasonic data from the transmission/reception circuit 32.

Note that when the reception band of the ultrasonic probe 3 is considerably lower than 1/2 of the sampling frequency of the A/D converter of the transmission/reception circuit 32, data can also be reduced in the A/D converter of the transmission/reception circuit 32 in the sampling direction to the extent that the sampling theorem is satisfied.

The aforementioned data thinning processing can also lower the transfer rate of the ultrasonic reception signal depending on the communication status. Furthermore, as needed, the aforementioned data thinning processing and a process of switching from the pre-beamforming data to the post-beamforming data may be combined.

Second Modification

In the above example, the case where the communication standard of the wireless terminal for data transfer 7 and the wireless I/F for data transfer 34 is single has been exemplified. On the other hand, a plurality of communication modes having different specifications may be provided on both the wireless terminal for data transfer 7 and the wireless I/F for data transfer 34, and communication may be performed in one or both of them in a parallel manner.

For example, if communication is possible with the 60-GHz band proposed in IEEE802.11ay and the 5-GHz band of the communication mode in the related art, even though one condition becomes worse, communication can be continued under the other condition.

Third Modification

In the aforementioned embodiment, the case where communication between the ultrasonic probe 3 and the wireless terminal for data transfer 7 as a relay device to the ultrasonic image server 2 is wirelessly performed, has been described as an example. On the other hand, the communication between the ultrasonic probe 3 and the relay device to the ultrasonic image server 2 may also be performed in a wired manner or in both wireless and wired manners.

When the communication between the ultrasonic probe 3 and the relay device to the ultrasonic image server 2 is performed in a wired manner, the wireless terminal for data transfer 7 is replaced with a wired terminal for data transfer and the wireless I/F for data transfer 34 is replaced with a priority I/F circuit for data transfer.

Furthermore, when the communication between the ultrasonic probe 3 and the relay device to the ultrasonic image server 2 is performed in parallel of wireless and wired manners, a wired terminal for data transfer is provided in the examination room R1 in addition to the wireless terminal for data transfer 7, and the ultrasonic probe 3 is configured to further include a wired I/F for data transfer in addition to the wireless I/F for data transfer 34.

Note that as the transfer rate of the present third modification, an example of wired communication capable of transferring (data rate: 8.3 Gbps) a compressed reception signal before beamforming processing as described above includes Ethernet (registered trademark) (10 GBase-T or higher), USB (3.1 Gen2 or higher), and InfiniBand (Quad Data rate or higher).

Fourth Modification

In the aforementioned embodiment, the case where at least one of the type of data transferred from the ultrasonic probe 3 and the communication speed between the ultrasonic probe 3 and the relay device is controlled on the basis of the diagnostic status and the communication status, has been described.

However, actually, the communication status may deteriorate due to various reasons. Furthermore, as for the degree of deterioration of the communication status, there may be a situation in which the minimum communication speed required for diagnosis is not able to be secured or a situation in which communication is not possible at all due to network failure and abnormalities in communication devices. As a result of the detection of the communication status by the server-side communication status detection function 262, when it is determined that the minimum communication speed required for diagnosis is not able to be secured or communication is not possible at all, the server-side control function 261 of the server-side control circuit 26 performs any one of issuing a warning on at least one of the ultrasonic image server 2 and the ultrasonic probe 3, controlling the ultrasonic probe 3 to stop scanning, displaying an image immediately before the situation in which the minimum communication speed required for diagnosis is not able to be secured, or the situation in which communication is not possible at all, on at least one of the examination room-side display circuit 50 and the server-side display circuit 23 as a still image, and stopping energy transmission from the scanner to a living body. Furthermore, on the basis of the diagnostic status and the communication status, the server-side control function 261 of the server-side control circuit 26 may determine whether at least one of a current imaging mode and an application is available, and output the determination result.

According to the configuration described above, even when the minimum communication speed required for diagnosis is not able to be secured or communication is not possible at all due to the deterioration of the communication status, it is possible to secure the best ultrasonic image display at that time.

Fifth Modification

In the first embodiment, when the ultrasonic probe 3 is wireless, the ultrasonic probe 3 needs to have a battery therein as a means for supplying power to the ultrasonic probe. In such a case, when the remaining battery level of the ultrasonic probe 3 is close to zero, it is regarded as the deterioration of the communication status and the same control as when the communication status deteriorates is performed, so that it is possible to secure the best ultrasonic image display at that time before the battery is exhausted and information required for diagnosis is not able to be communicated at all.

### Second Embodiment

In the first embodiment, the server-side communication status detection function 262 monitors a communication status between the ultrasonic image server 2 and the ultrasonic probe 3 connected to the ultrasonic image server 2 to perform data transfer, and measures an upper limit of a data transfer rate that can be effectively achieved. In such a case, the detection by the server-side communication status detection function 262 needs to be performed with high frequency.

On the other hand, the communication status mainly depends on the position of the ultrasonic probe 3. Therefore, an ultrasonic diagnostic system SG according to the second embodiment measures the position of the ultrasonic probe 3 and detects the communication status on the basis of the measurement result.

The ultrasonic diagnostic system SG acquires in advance a communication status for each position of the ultrasonic probe 3 inside the examination room R1 as communication information, and stores the communication information in a storage circuit 124. The server-side control circuit 26 can detect a communication status between the ultrasonic probe 3 and the wireless terminal for data transfer 7 in real time by referring to the position of the ultrasonic probe 3 measured in real time and the communication information read from the storage circuit 124 at the time of examination by the server-side control function 261, and use the detected communication status for comparison with a diagnostic status. Accordingly, it is not necessary to detect the communication status with high frequency.

Note that the positioning of the ultrasonic probe 3 can be implemented, for example, by using a terminal having a positioning function such as Bluetooth (registered trademark) as the wireless terminal for control 6. As the positioning method in such a case, for example, it is possible to adopt an RSSI (signal strength) method in which the position of a receiver is estimated from the radio wave intensity of a transmitter, an angle of arrival (AoA) method in which the angle of arrival of radio waves is also measured as well as the radio wave intensity, and a triangulation positioning method in which positioning is performed using a triangulation method by means of a plurality of transmitters.

Other methods for the positioning of the ultrasonic probe 3 include a method in which a magnetic generator is placed in the vicinity of the ultrasonic probe 3 and a magnetic sensor attached to the ultrasonic probe 3 side is used, a method using an acceleration sensor, a method of imaging the ultrasonic probe 3 with an optical camera and calculating the position of the ultrasonic probe 3, a method using a global positioning system (GPS), and the like, and a plurality of methods can also be combined in order to increase the positioning accuracy. Furthermore, the positioning dimension may not be limited to two dimensions and may include a three-dimensional space including height.

When the positioning of the ultrasonic probe 3 is possible, whether the ultrasonic probe 3 is located in a predetermined examination area is determined by the position thereof, and when it is determined that the ultrasonic probe 3 is out of the examination area, it is possible to obtain the best display at that time by performing any one of issuing a warning, stopping scanning, displaying an image immediately before the determination on a display means as a still image, and stopping energy transmission from the scanner to a living body.

### Third Embodiment

In the first and second embodiments, the case where the combination of the ultrasonic probe 3, the ultrasonic image server 2, the examination room-side input I/F circuit 40, and the examination room-side display circuit 50 which are connected to the network, constitutes one ultrasonic diagnostic system SG has been described as an example. On the other hand, the following description will be given for a case where an ultrasonic diagnostic system SG according to the third embodiment is a combination of a portable ultrasonic diagnostic device in which the examination room-side input I/F circuit 40 and the examination room-side display circuit 50 are also mechanically incorporated in a main body, and an ultrasonic probe, and the device is taken out from a hospital to a remote place.

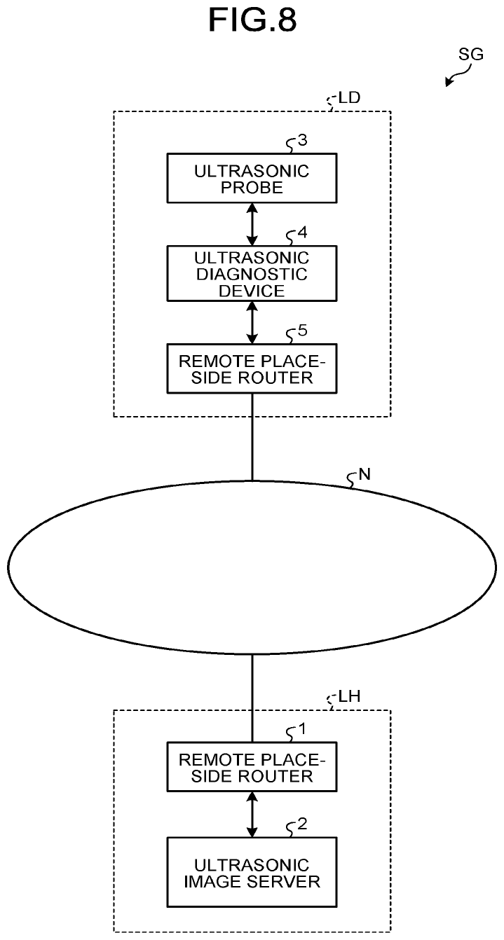

FIG. 8 is a diagram illustrating the configuration of the ultrasonic diagnostic system SG according to the third embodiment. As illustrated in FIG. 8, the ultrasonic diagnostic system S is configured by an ultrasonic probe 3, an ultrasonic diagnostic device 4 as a portable ultrasonic diagnostic device, and a remote place-side router 5 which are provided in a remote place LD, and a hospital-side router 1 and an ultrasonic image server 2 provided in a hospital. The remote place-side router 5 and the hospital-side router 1 can communicate with each other via the network N as a public line. Note that the hospital-side router 1 and the ultrasonic image server 2 do not necessarily have to be installed in the hospital, and may be installed anywhere as long as they can communicate with the remote place-side router 5 via the network N.

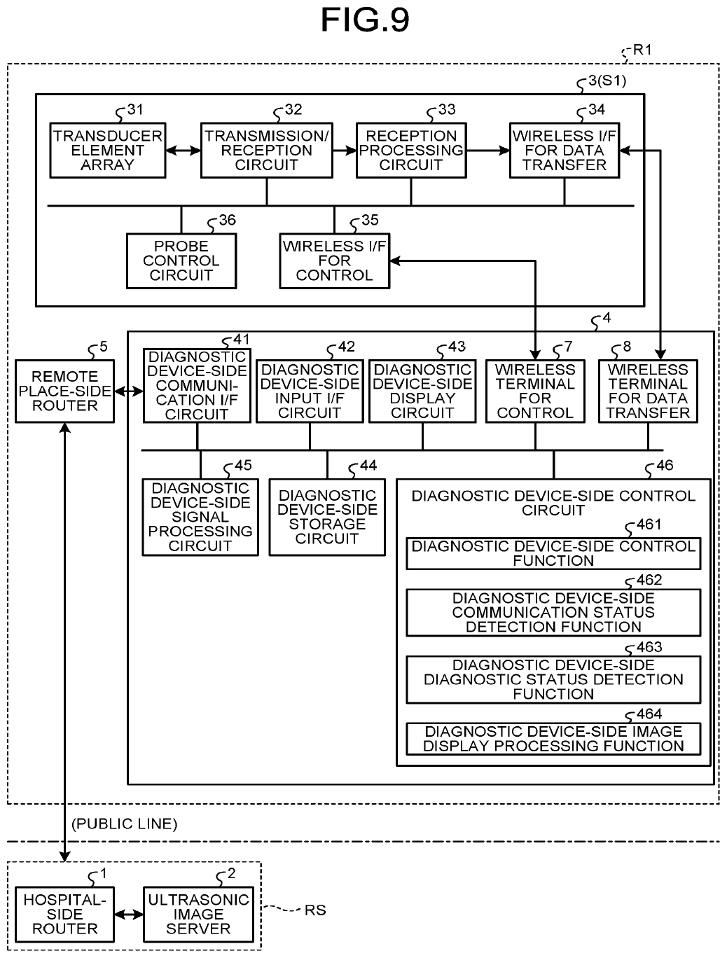

FIG. 9 is a block diagram illustrating configurations of the ultrasonic probe 3, the ultrasonic diagnostic device 4, the ultrasonic image server 2 included in the ultrasonic diagnostic system SG according to the third embodiment. Hereinafter, the configuration of the ultrasonic diagnostic device 4 will be described with reference to FIG. 9. Note that since the configurations of the ultrasonic image server 2 and the ultrasonic probe 3 are the same as those illustrated in FIG. 2, a description thereof will be omitted.

The ultrasonic diagnostic device 4 receives ultrasonic data from the ultrasonic probe 3 by, for example, wireless communication, and performs signal processing and the like on the received ultrasonic data to generate an ultrasonic image. More specifically, the ultrasonic diagnostic device 4 includes a diagnostic device-side communication I/F circuit 41, a diagnostic device-side input I/F circuit 42, a diagnostic device-side display circuit 43, a diagnostic device-side storage circuit 44, a diagnostic device-side signal processing circuit 45, a diagnostic device-side control circuit 46, a wireless terminal for control 6, and a wireless terminal data transfer 8. Furthermore, the diagnostic device-side control circuit 46 includes a diagnostic device-side control function 461, a diagnostic device-side communication status detection function 462, a diagnostic device-side diagnostic status detection function 463, and a diagnostic device-side image display processing function 464.

The diagnostic device-side communication I/F circuit 41, the diagnostic device-side input I/F circuit 42, the diagnostic device-side display circuit 43, the diagnostic device-side storage circuit 44, the diagnostic device-side signal processing circuit 45, the diagnostic device-side control circuit 46, the diagnostic device-side control function 461, the diagnostic device-side diagnostic status detection function 463, and the diagnostic device-side image display processing function 464 have substantially the same configurations as those of the server-side communication I/F circuit 21, the examination room-side input I/F circuit 40, the examination room-side display circuit 50, the server-side storage circuit 24, the server-side signal processing circuit 25, the server-side control circuit 26, the server-side control function 261, the server-side diagnostic status detection function 263, and the server-side image display processing function 264, respectively.

The diagnostic device-side communication status detection function 462 of the ultrasonic diagnostic device 4 and the server-side communication status detection function 262 of the ultrasonic image server 2 detect a communication speed between the ultrasonic probe 3 and the ultrasonic diagnostic device 4, a communication speed between the ultrasonic diagnostic device 4 and the remote place-side router 5, a communication speed between the hospital-side router 1 and the ultrasonic image server 2, a communication speed between the ultrasonic diagnostic device 4 and the ultrasonic image server 2 (that is, between the remote place-side router 5 and the hospital-side router 1).

On the basis of a communication status and a diagnostic status, the diagnostic device-side control function 461 of the ultrasonic diagnostic device 4 controls at least one of the output switching circuit 333 of the reception processing circuit 33, the communication speed between the ultrasonic probe 3 and the ultrasonic diagnostic device 4, and the communication speed between the ultrasonic diagnostic device 4 and the remote place-side router 5.

On the basis of the communication status and the diagnostic status, the server-side control function 261 of the ultrasonic image server 2 controls at least one of the output switching circuit 333 of the reception processing circuit 33, the communication speed between the ultrasonic probe 3 and the ultrasonic diagnostic device 4, the communication speed between the ultrasonic diagnostic device 4 and the remote place-side router 5, the communication speed between the hospital-side router 1 and the ultrasonic image server 2, and the communication speed between the ultrasonic diagnostic device 4 and the ultrasonic image server 2.

The wireless terminal for control 6 is connected to the wireless I/F for control 35 and transmits/receives a control signal converted into a wireless signal conforming to the wireless communication standard. The control signal is exchanged between the probe control circuit 36 and the ultrasonic diagnostic device 4 via the wireless terminal for control 6. Furthermore, the control signal of the ultrasonic probe 3 can communicate separately with the wireless terminal for control 6 by a wireless communication mode different from that for an ultrasonic reception signal. Furthermore, the wireless terminal for control 6 exchanges the control signal with the ultrasonic image server 2 via the remote place-side router 5 and the hospital-side router 1.

The wireless terminal data transfer 8 is connected to the wireless I/F for data transfer 34, and receives an ultrasonic reception signal before beamforming processing or an ultrasonic reception signal after beamforming processing which has been converted into a wireless signal conforming to the wireless communication standard. Furthermore, the wireless terminal data transfer 8 exchanges ultrasonic data and ultrasonic image data with the ultrasonic image server 2 via the remote place-side router 5 and the hospital-side router 1.

In the ultrasonic diagnostic system SG, for example, communication conditions are different between the ultrasonic probe 3 and the ultrasonic diagnostic device 4, between the ultrasonic diagnostic device 4 and the remote place-side router 5, between the hospital-side router 1 and the ultrasonic image server 2, and between the ultrasonic diagnostic device 4 and the ultrasonic image server 2 (that is, between the remote place-side router 5 and the hospital-side router 1). In general, since a communication path between the ultrasonic diagnostic device 4 and the ultrasonic image server 2 includes public communication, conditions related to data transfer are strict as compared with the ultrasonic diagnostic system SG according to the first embodiment, for example.

Even in such a communication environment, in accordance with the ultrasonic diagnostic system SG according to the present embodiment, it is possible to change at least one of a signal to be output by signal processing and the communication speed of the communication unit according to a change in the diagnostic status and the communication status. Accordingly, similarly to the first and second embodiments, even when the examination or communication status is changed, it is possible to continue and maintain the diagnostic function as much as possible.

Fifth Modification

For example, as a further modification of the third embodiment, a configuration example in which the ultrasonic probe 3 and the ultrasonic diagnostic device 4 are mechanically integrated is conceived. Furthermore, as a further modification, the ultrasonic diagnostic device may be a stationary type instead of a portable type, and may be installed in an examination room instead of a remote place.

According to at least one embodiment described above, in an environment in which data communication is performed between the scanner and the image processing server via the network, even when the diagnostic status or the communication status is changed, it is possible to implement stabler operations than the related art.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image diagnostic system comprising:
a first device that acquires a first signal to be used for diagnosing a subject, acquires a second signal from the first signal, and outputs the first signal or the second signal;
a second device including a generation unit that generates image data by using the first signal or the second signal;
a communication circuit that establishes communication between the second device and at least the first device, and transfers the first signal or the second signal that is output from the first device, to the second device;
at least one first detection circuit that detects a communication status related to the communication from the communication unit;
at least one second detection circuit that detects a diagnostic status related to the subject from the first device; and
a control circuit that controls at least one of the output from the first device and a communication speed of the communication circuit on the basis of the diagnostic status and the communication status.

2. The medical image diagnostic system according to claim 1, wherein the first device is an ultrasonic probe, and the second device is an ultrasonic image server including the first detection circuit, the second detection circuit, and the control circuit.

3. The medical image diagnostic system according to claim 1, wherein the first device is an ultrasonic diagnostic device including an ultrasonic probe, the first detection circuit, the second detection circuit, and the control circuit, and the second device is an ultrasonic image server including the first detection circuit, the second detection circuit, and the control circuit.

4. The medical image diagnostic system according to claim 2, wherein the control circuit acquires a first data rate required for transferring the first signal from the first device to the second device and a second data rate required for transferring the second signal from the first device to the second device, on the basis of the diagnostic status,
acquires a maximum data rate in the communication on the basis of the communication status, and
controls at least one of the output from the first device and the communication speed of the communication unit on the basis of the first data rate, the second data rate, and the maximum data rate.

5. The medical image diagnostic system according to claim 3, wherein the control circuit acquires a first data rate required for transferring the first signal from the first device to the second device and a second data rate required for transferring the second signal from the first device to the second device, on the basis of the diagnostic status,
acquires a maximum data rate in the communication on the basis of the communication status, and
controls at least one of the output from the first device and the communication speed of the communication unit on the basis of the first data rate, the second data rate, and the maximum data rate.

6. The medical image diagnostic system according to claim 2, wherein the first detection circuit detects the diagnostic status on the basis of at least one of a current imaging mode in the first device, a type of the ultrasonic probe, and an imaging condition.

7. The medical image diagnostic system according to claim 2, wherein the first signal is a signal before beamforming, and
the second signal is a signal after beamforming.

8. The medical image diagnostic system according to claim 2, wherein the control circuit determines a priority related to communication between the first device and the second device on the basis of the diagnostic status, and
exclusively establishes the communication between the first device and the second device according to the priority.

9. The medical image diagnostic system according to claim 2, wherein, when the control circuit determines that a communication speed for transferring the second signal from the first device to the second device is not able to be secured, the control circuit performs at least one of output of a warning, stop of imaging by the first device, and stop of energy transmission from the first device to the subject, on the basis of the diagnostic status and the communication status.

10. The medical image diagnostic system according to claim 2, further comprising:
a measurement unit that measures a position of the ultrasonic probe, wherein
the second detection circuit detects the communication status on the basis of the position of the ultrasonic probe.

11. The medical image diagnostic system according to claim 9, wherein the control circuit determines whether at least one of a current imaging mode and an application is available on the basis of the diagnostic status and the communication status, and outputs a result of the determination.

12. The medical image diagnostic system according to claim 2, further comprising:
a measurement unit that measures a position of the ultrasonic probe, wherein
when the position of the ultrasonic probe is a position where imaging of the subject is not possible, the control circuit performs at least one of output of a warning, stop of imaging by the first device, and stop of energy transmission from the first device to the subject.

13. An ultrasonic probe that performs communication with an ultrasonic image server, the ultrasonic probe comprising:
a plurality of ultrasonic transducer elements that transmit ultrasonic waves to a subject according to a supplied driving signal and receive reflected waves from the subject;
a reception unit that generates a first signal on the basis of output from the ultrasonic transducer elements;
a reception processing unit that acquires a second signal from the first signal and outputs the first signal or the second signal;
a switching unit that switches the output of the reception processing unit on the basis of a control signal from the ultrasonic image server; and
a transfer unit that transfers the output of the reception processing unit to the ultrasonic image server.

14. A medical image diagnostic system comprising:
a server; and
a plurality of ultrasonic probes wirelessly connected to the server, wherein
the server determines allocation of a communication band to the ultrasonic probes according to diagnosis performed by the ultrasonic probes.

15. A medical image diagnostic device comprising:
a communication circuit that acquires a first signal to be used for diagnosing a subject, acquires a second signal from the first signal, establishes communication with the first device that outputs the first signal or the second signal, and transfers the first signal or the second signal that is output from the first device, to the second device;
a generation circuit that generates image data by using the first signal or the second signal;
at least one first detection circuit that detects a communication status related to the communication from the communication unit;
at least one second detection circuit that detects a diagnostic status related to the subject from the first device; and
a control circuit that controls at least one of the output from the first device and a communication speed of the communication unit on the basis of the diagnostic status and the communication status.
